(19) United States
(12) Patent Application Publication
(10) Pub. No.: US 2025/0082950 A1
(43) Pub. Date: Mar. 13, 2025

(54) CARDIAC ARREST MONITOR AND AED INTEGRATION

(71) Applicant: Avive Solutions, Inc., Brisbane, CA (US)

(72) Inventor: Rory M. BEYER, San Mateo, CA (US)

(21) Appl. No.: 18/882,131

(22) Filed: Sep. 11, 2024

Related U.S. Application Data
(60) Provisional application No. 63/581,902, filed on Sep. 11, 2023.

(51) Int. Cl. A61N 1/39; A61N 1/02; H04L 67/12

(52) U.S. Cl. CPC A61N 1/3925; A61N 1/025; A61N 1/3904; A61N 1/3993; H04L 67/12

(57) ABSTRACT

A variety of AEDs and AED/cardiac monitor integrations are described. In some embodiments, the AED is configured to wirelessly receive incident notifications from a cardiac monitor indicating that the monitor has detected a cardiac arrest in a monitored patient. In response to reception of the incident notification, the AED generates an auditory and/or visual incident alert to inform nearby individuals of the potential cardiac arrest. The AED also sends a second incident notification to a remotely located server to inform the server of the incident. In another aspect, a kit that includes both an AED and a cardiac monitor is described. The kit is configured to facilitate sending incident notifications to the AED either directly or via one or more intermediate servers to cause the AED to generate the incident alert.

### CROSS-REFERENCE TO RELATED APPLICATIONS

This application claims priority of U.S. Provisional Application No. 63/581,902, filed on Sep. 11, 2023, which is incorporated herein by reference in its entirety.

### FIELD

The present disclosure relates generally to frameworks for integrating cardiac arrest monitors with AEDs.

### BACKGROUND

Sudden cardiac arrest is one of the leading causes of death. In the United States alone, roughly 350,000 people die each year from sudden cardiac arrest. It is the leading cause of death for individuals over 40 and the #1 killer of student athletes. The most effective treatment for sudden cardiac arrest is the use of CPR coupled with defibrillation. Automated external defibrillators (AEDs) are portable devices designed to automatically check for life-threatening heart rhythms associated with sudden cardiac arrest and to send an electrical shock to the heart to try to restore a normal rhythm when shockable heart rhythms are detected. The two most common conditions treated by AEDs are Pulseless Ventricular tachycardia (aka VT or V-Tach) and Ventricular fibrillation (VF or V-Fib). AEDs are typically designed so that they can be used by a lay person in situations where professional medical help is not available.

Given their potential to save lives, automated external defibrillators have been deployed in a relatively wide variety of public and private locations so that they are available in the event that a person in the vicinity goes into cardiac arrest. By way of example, AEDs may be found in corporate and government offices, shopping centers, airports, airplanes, restaurants, casinos, hotels, sports stadiums, schools, fitness centers and a variety of other locations where people may congregate.

Some individuals are known to be particularly at risk for cardiac arrest. That may be due to a history of arrythmias, a low heart ejection fraction, or other known medical conditions. When the risk is high enough, a patient may be given a surgical implant known as an implantable cardioverter-defibrillator (ICD). There are often waiting times associated with ICDs, so if the patient's risk profile is high enough, they may be prescribed a wearable cardioverter defibrillator such as the Zoll LifeVest that can both identify shockable heart rhythms and deliver defibrillation and/or cardioversion shocks when and as necessary. Although commercially available wearable cardioverter defibrillators have the capacity to save lives, they also have several drawbacks. Initially, they are very expensive and therefore tend to only be prescribed in extreme high-risk cases. Furthermore, many patients find the wearable defibrillators to be very uncomfortable and thus even when they are prescribed, the patient simply stops wearing the device when they should.

There are also many patients that have an elevated risk for cardiac arrest, but their risk is not deemed high enough to be eligible for insurance reimbursement for implantable cardioverter defibrillators, pacemakers, or wearable defibrillators or for other reasons simply don't have access to such technologies.

In some situations, a cardiac monitor may be prescribed for longer term monitoring of the heart's electrical activity thereby facilitating the automatic detection of arrythmias, tachycardia and/or other abnormal heart rhythms. Such monitors include, but are not limited to, wearable Holter monitors and implantable cardiac monitors (ICM). These cardiac monitors can be particularly useful in detecting and identifying infrequent and/or unexplained heart rhythm abnormalities. Other bioelectronic monitors (wearable, implantable or otherwise) may be arranged to monitor a variety of physiological parameters related to heart function in addition to/or instead of the patient's electrocardiogram (ECG). Implantable monitors have advantages over wearable monitors in that they typically can't be removed by the patient and tend to be best suited for longer term monitoring. Conversely, Holter monitors have significant cost advantages and are particularly well suited for shorter term monitoring—e.g., monitoring over a period of days rather than months. Although cardiac monitors are well suited for identifying abnormal and life-threatening heart rhythms, they do not have the ability to treat life threatening events such as cardiac arrest.

In any of the above situations in which a patient does not have an ICD, if the patient experiences a cardiac arrest, rapid deployment of an AED may be their best chance of survival. Although existing AEDs work well, most cardiac arrests occur at home where AEDs are less common, and many are unwitnessed/unnoticed and therefore are untreated in the short window where intervention can be helpful. Therefore, there are continuing efforts to develop programs and devices that improve the probability that AEDs will be available and used when an out-of-hospital cardiac arrest occurs.

### SUMMARY

A variety of cardiac arrest monitor/AED integrations are described. In one aspect an AED includes a receiver configured to wirelessly receive a first incident notification transmitted by a cardiac arrest monitor indicating that the monitor has detected a potential cardiac arrest in a patient monitored by the monitor. In response to reception of the first incident notification, the AED generates a human perceptible incident alert to inform individuals that are nearby both the monitor and the AED of the potential cardiac arrest, to thereby encourage such individuals to respond to the potential cardiac arrest incident using the AED. The AED also sends a second incident notification to a remotely located server to thereby inform the remotely located server of the potential cardiac arrest incident. The second incident notification is sent in real time relative to the reception of the first incident notification.

In some embodiments, the AED includes a speaker, and the human perceptible incident alert is or includes an auditory alert generated by the speaker. In some embodiments, the AED includes a display screen, and the human perceptible incident alert is or includes a visual alert displayed on the display screen.

In some embodiments, the remotely located server is a volunteer responder network server or an AED management server.

In some embodiments, the receiver that receives the first incident notification is a Bluetooth Low Energy (BLE) receiver. In some embodiments, the first incident notification is a broadcast by the cardiac monitor as either a beacon or a BLE advertisement. In some embodiments, the AED includes a BLE transmitter, and the AED is further configured to serve as a repeater that rebroadcasts the first incident notification using the BLE transmitter.

In general, the AED has one or more processors, one or more memories and programmed instructions in the form of software code (which may be or include firmware) stored in the one or more memories that is configured to direct the described functionalities.

In another aspect, a cardiac arrest detection and treatment kit is described. The kit includes a cardiac arrest monitor and an AED. The monitor has a transmitter and is configured to detect a potential cardiac arrest in a patient monitored by the cardiac monitor. If/when a potential cardiac arrest is detected, a controller on the monitor causes the monitor transmitter to wirelessly transmit a first incident notification. The AED includes a first communication unit configured to communicate with a remotely located server and a second communications unit configured to at least one of send or receive short-range wireless communications. The AED is also configured to wirelessly receive at least one of the first incident notification transmitted by the cardiac monitor or a second incident notification received from the remotely located server that is directly or indirectly triggered by the first incident notification. The AED is further configured to, in response to reception of the first or second incident notification, generate a human perceptible incident alert to inform individuals that are nearby the AED associated with the monitor of the potential cardiac arrest to thereby encourage such individuals to respond to the potential cardiac arrest incident using the AED. Still further, the AED is configured to communicate with the remotely located server regarding the potential cardiac arrest incident.

In some embodiments, the monitor is a cardiac monitor, as for example a Holter monitor, an implantable cardiac monitor (ICM) or an ECG/EKG monitor. In other embodiments the monitor may take the form of other devices capable of detecting potential cardiac arrest incidents such as monitors that include fall detectors, agonal breathing detectors, etc.

In some embodiments, the kit is associated with an insurance reimbursement code.

In some embodiments of the kit, the AED is configured to receive and act upon both first incident notifications and second incident notifications. In others, the AED is only capable of acting on one of the first and second incident notifications and not the other.

In some embodiments of the kit, the remotely located server is an AED management server. The first incident notification is sent directly or indirectly from the cardiac monitor to a monitor management server, which sends a third incident notification directly or indirectly to the AED management server. In response to the reception of the third incident notification, the AED management server directly or indirectly sends the second incident notification to the AED.

In some embodiments of the kit, the AED is configured to receive the first incident notification. In response thereto, the AED sends a third incident notification to the remotely located server to thereby inform the remotely located server of the potential cardiac arrest incident. The third incident notification being sent in real time relative to the reception of the first incident notification.

In some embodiments, the cardiac monitor is selected from the group consisting of a Holter monitor, an implantable cardiac monitor (ICM) and an ECG/EKG monitor.

In some embodiments, the cardiac monitor includes a Bluetooth Low Energy (BLE) transmitter that transmits the first incident notification, and the AED includes a BLE receiver that receives the first incident notification.

In some embodiments of the first incident notification is broadcast as one selected from the group consisting of a beacon and an advertisement, such as a BLE advertisement.

In general, the AED has one or more processors, one or more memories and programmed instructions in the form software code (which may be or include firmware) stored in the one or more memories that is configured to direct the described AED functionalities. Similarly, the cardiac monitor has one or more processors, one or more memories and programmed instructions stored in the one or more monitor memories that is configured to direct the described monitor functionalities.

In another aspect, an AED includes a shock delivery circuit capable of delivering a defibrillation shock, one or more processors, one or more memories that is/are accessible by the processors and a receiver configured to wirelessly receive a beacon transmitted by a cardiac monitor indicating that the cardiac monitor has detected a potential cardiac arrest in a patient monitored by the cardiac monitor. The AED further includes programed instruction stored in the one or more memories that includes beacon handling code and incident alert code. The beacon handling code is configured to decode the beacon. The incident alert code is configured to, in response to reception of the beacon, cause the AED to generate a human perceptible incident alert to inform individuals that are nearby both the cardiac monitor and the AED of the potential cardiac arrest to thereby encourage such individuals to respond to the potential cardiac arrest incident using the AED.

In yet another aspect, the AED is configured to serve as a relay that forwards incidents alerts received from a cardiac monitor to other nearby AEDs.

### BRIEF DESCRIPTION OF THE DRAWINGS

The invention and the advantages thereof, may best be understood by reference to the following description taken in conjunction with the accompanying drawings in which:

FIG. 1 is a block diagram showing a cardiac arrest detection and treatment kit in accordance with a first embodiment.

FIG. 2 is a flow chart illustrating a representative use case for the cardiac arrest detection and treatment kit.

FIG. 3 is a block diagram of an emergency response system that integrates the cardiac arrest detection and treatment kit.

FIG. 4 is a diagrammatic representation of a modular defibrillator system architecture.

FIG. 5 is a block diagram illustrating electrical components of a representative defibrillator unit.

FIG. 6 is a block diagram illustrating electrical components of a representative interface unit.

FIG. 7 is a diagrammatic illustration of several system architectures suitable for notifying an AED of a cardiac arrest detected by a cardiac monitor.

In the drawings, like reference numerals are sometimes used to designate like structural elements. It should also be appreciated that the depictions in the figures are diagrammatic and not to scale.

### DETAILED DESCRIPTION

The present disclosure describes cardiac arrest detection monitors and AED integration frameworks that may be helpful in improving cardiac arrest survival statistics. In some embodiments, a cardiac arrest detection and treatment kit is provided that include a cardiac monitor capable of identifying a potential cardiac arrest together with a separate automated external defibrillator (AED) capable of delivering a defibrillation shock to the cardiac arrest victim. In another aspect, frameworks for communicating incident notifications between cardiac monitors and AEDs are described.

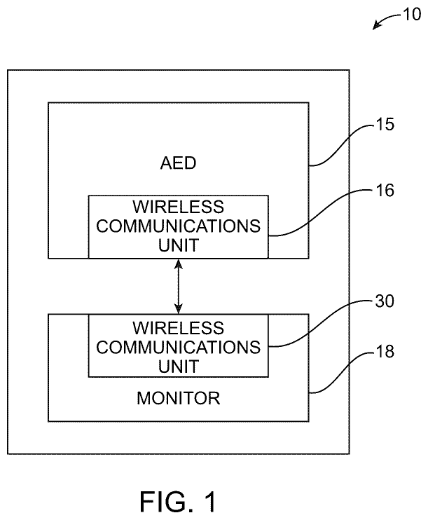

Referring initially to FIG. 1, a cardiac arrest preparedness kit 10 in accordance with one embodiment includes an AED 15 (which is preferably a connected AED) and a wearable or implantable monitoring device (monitor 18) that is capable of identifying a potential cardiac arrest or sensing biometric information that is indicative of cardiac arrest. In some embodiments, the AED and the monitor are packaged together in a physical kit as illustrated in FIG. 1. In other embodiments, the AED and the monitoring device may be prescribed together although they may be sold and/or delivered separately. In still other embodiments, the AED and the monitoring device may simply be designed to work together so that they may be deployed and used in the described manners.

The monitor 18 may take a wide variety of forms and in some embodiments more than one type of monitor may be included, or the monitor may be capable of sensing multiple biometric parameters. In some embodiments, the monitor is a wearable device (e.g., a Holter monitor, or an ECG/EKG monitor) or an implantable device (e.g., an implantable cardiac monitor (ICD)) that monitors the patient's ECG and heart rhythm. Although monitors that can monitor at least the patient's ECG and/or heart rhythms are generally preferred, other types of monitors capable of detecting potential cardiac arrest incidents, including fall detectors, agonal breathing detectors and others, may be used in other embodiments.

Wearable monitors may take the form of a vest, a band, a patch, a plurality of electrodes or any other suitable form. For example, in some embodiments, the wearable monitor is worn around or otherwise applied to the chest, while in others it fits on the user's arm, wrist, leg, neck or other suitable location. In some embodiments, a wearable monitor is a multipurpose device that has a variety of functionalities beyond serving as a heart monitor such as a smart watch, a fitness tracker, a fall detector, a glucose monitor, etc.

Implantable cardiac monitors (ICMs) are typically designed to continuously monitor the patient's electrocardiogram (ECG) and heart rate. One common type of ICM is the implantable loop recorder (ILR). However, ICMs may have a variety of additional or different sensors that gather other health related information or supplemental information that may be useful in analyzing the ECGs or for diagnostic purposes. Other sensors may include acceleration sensors, glucose monitors, pulse oximeters, etc. Of course, these types of other sensors and others may also be included in wearable monitors.

The AED 15 is preferably a connected AED that is capable of communicating with external devices using multiple communications pathways such as Bluetooth/Bluetooth Low Energy (BLE), Wi-Fi and cellular communications. In the embodiment illustrated in FIG. 1, these communication capabilities are provided by wireless communications unit 16. In other embodiments additional and/or alternative communications capabilities may be included, as for example, satellite communications, various short-range communications technologies (e.g., Wi-Fi Direct, Near Field Communications (NFC), Bluetooth, Zigbee, Z-Wave, Ultra-Wideband (UWB) etc.), and others, including proprietary communications protocols.

The Applicant has developed defibrillators that incorporate a number of connectivity features including Bluetooth/Bluetooth Low Energy (BLE), Wi-Fi and cellular communications that make them particularly well suited for use in conjunction with cardiac monitors and other devices that are capable of detecting cardiac arrest. By way of example, U.S. Pat. No. 11,534,618 (P016BC1); U.S. Pat. No. 11,452,881 (P016A); U.S. Pat. No. 10,773,091 (P006E); and U.S. Pat. No. 10,737,105 (P006A), each of which is incorporated herein by reference, describe a few such connected defibrillators that are well suited for use as AED 15.

In some embodiments, the monitor 18 has a wireless communications unit 30 that facilitates wireless communications with external devices, including, but typically not limited to the AED 15. Wireless communications unit 30 may support one or more communications protocols. Typically, the supported communications protocol(s) will be short-range communication protocol such as Bluetooth/Bluetooth Low Energy (BLE), Wi-Fi/Wi-Fi Direct, Near Field Communications (NFC), Zigbee, Z-Wave, Ultra-Wideband (UWB), proprietary short-range protocols, etc. However, in other embodiments, other and longer-range protocols may also be supported.

In some embodiments, the monitor 18 includes, or has an associated base unit (e.g., a base station). The base unit may be arranged to be worn or carried together with a separate implantable or wearable sensing unit. Such base units can extend the functionality of the monitor. In some implementations, the base unit is a dedicated base unit whose sole function is to work in conjunction with the sensing unit. In other implementations, the base unit may have other functions as well. For example, in some implementations, the patient's cell phone (e.g., a Smartphone) may be used as the base unit. In other implementations the base unit is intended to remain at a home location (e.g., the patient's home) and the monitor synchronizes its data when the patient returns home. In such implementations, it is preferred that the AED 15 can directly receive incident notification 405 from the sensing unit itself.

There are a number of existing monitors (both wearable and implantable) that may be used as monitor 18 and such monitors have a wide variety of functionalities, and it is expected that new cardiac monitors having additional and/or improved functionalities will be developed in the future. Such monitors can be utilized in kit 10 without degrading their other functionalities. Since a wide variety of such monitors are known in the art, we do not try to articulate all of their various capabilities. Rather, this disclosure focuses on the integration of such monitors with the AED 15, which can be facilitated by the kit. Although this description refers primarily to implanted and wearable monitors, it should be appreciated that in other embodiments, the monitor may have sensors that do not require contact with the patient (e.g., camera-based monitoring) or may otherwise take or include form factors that are neither wearable nor implantable.

When the patient is at risk of cardiac arrest, it is advisable for the AED 15 to be kept relatively close to the patient at all times. For example, when the patient is at home, it would be expected that the AED 15 be available in the home. If the patient is out of the house or traveling, it would be expected that the AED would accompany the patient. If the patient is at work, it would be expected that an AED would be available at work. Although this discussion is based on a scenario where a single AED (e.g., AED 15) is carried around or otherwise kept near the patient, it should be appreciated that in alternative scenarios, different AEDs may be provided in different locations. For example, one AED may be kept at home, another at the office and a third in a carrying bag that the patient carries with them when not at home or another location that has an AED nearby.

When a patient is in cardiac arrest, they are past the point of being able to self-help. Therefore, if/when the monitor 18 detects that the patient is in cardiac arrest, the monitor directly or indirectly sends an incident notification to the AED 15 that causes the AED to generate an incident alert. The incident alert may include auditory and/or visual components, as for example a loud audible message stating that the patient is in cardiac arrest and asking for bystander assistance in using the AED to try to treat the cardiac arrest. When the AED has a display screen, the incident alert may also include a visual alert displayed (in some embodiments flashing) on the display screen. If the AED has other alerting functionalities (e.g., tactile notifications) the incident alert may include such functionalities as well.

It should be appreciated that the incident alerts generated by the AED provide several benefits. For example, in some circumstances, there may be people near the patient that could help but aren't even aware that the patient is in cardiac arrest (e.g., the victim is in another room of a house, or building or otherwise not visible to family members, colleagues or bystanders). Even if someone is present near the victim, they may not be aware that the patient is in distress—for example they may be asleep or assume that the victim is asleep or resting—or they may simply not know what to do even if they can see that the victim is in distress. Most cardiac arrests occur at home, and many are unwitnessed, or family members aren't aware of the situation until it is too late. Thus, incident alerts generated by the AED can be particularly helpful in notifying family members (or more generally housemates, bystanders, etc.) of the emergency, as well as providing a tool that can be used to begin treatment before traditional emergency medical services (EMS) arrive on the scene. In still other circumstances, a bystander may be aware of the medical emergency, but may be unaware that an AED is located nearby, and the incident alert generated by the AED may bring the AED to the bystander's attention so that it can be retrieved and utilized prior to the arrival of EMS.

It should be appreciated that many cardiac monitors have their own emergency response protocols. For example, some monitors are configured to send incident messages to friends or family to prompt a response, some may generate an audible alert that bystanders can hear to make them aware of the incident, still others try to integrate a medical or emergency response. Preferably, the AED incident notifications are provided in parallel to such monitor functionalities and not in place of any such services provided by the monitor.

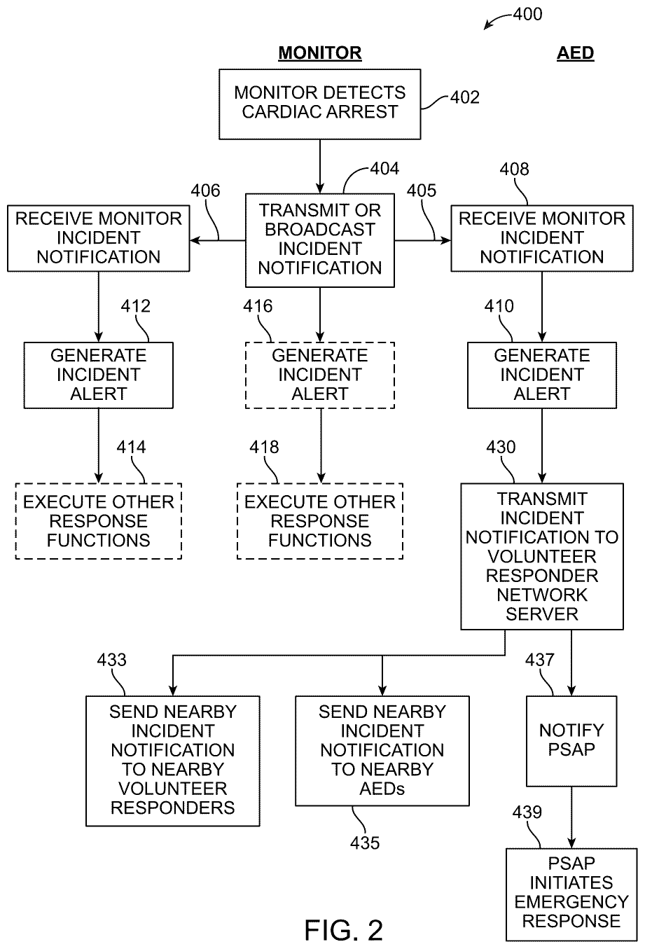

Referring next to the flow chart 400 of FIG. 2, some representative use cases for the cardiac arrest detection and treatment kit will be described. In the described embodiment, the monitor 15 is an implantable or wearable monitor (e.g., an implantable cardiac monitor or a wearable Holter monitor, or an ECG/EKG monitor) that records the patient's ECG and is capable of identifying heart rhythms indicative of cardiac arrest. In other embodiments, other types of monitors capable of detecting that the patient is experiencing cardiac arrest may be used. If/when the monitor detects cardiac arrest (block 402), it transmits an incident notification to at least the affiliated AED 15. Block 404. In some embodiments, the AED may be the only device that receives an incident alert. In others embodiments, incident notifications may also be sent to other devices, as for example, Smart Phones or other devices of family, friends, attendants, etc.; a base station/unit etc. as represented by arrow 406.

The incident notifications sent by the monitor may vary widely based on design goals. In some preferred embodiments, incident notifications include one or more of: (i) an identifier that uniquely identifies the reporting monitor (e.g., a unique monitor ID); (ii) the time that the potential cardiac arrest was detected (e.g., an incident timestamp); (iii) an incident identifier (which in some implementations may simply be a concatenation of the monitor ID and the incident timestamp); (iv) the geo-location of the incident (e.g., the location of the wearable or implantable device itself if available, or the location of a base station that is affiliated with the monitor), preferably together with a location accuracy measurement; (v) a patient identifier that identifies the patient associated with the monitor; (vi) a classification of the sensed cardiac rhythm; (vii) current status indicator that indicates a current status of the event (e.g., an active arrest, have they exited or degraded, treatment in progress, etc.)—this is particularly valuable when the monitor updates or repeatedly broadcasts or otherwise sends incident notifications; (viii) an incident descriptor that indicates the nature of the incident that has been detected (e.g., for a cardiac monitor this can be the type of cardiac arrhythmia detected by a cardiac monitor, for fall based detectors, this can be the detection of a fall and unresponsiveness of the patient by the monitor, etc.) (viii) a current status indicator that indicates the current status of the event, etc. (e.g., an active arrest, and expired arrest, treatment in progress, etc.); and/or other information available to the monitor.

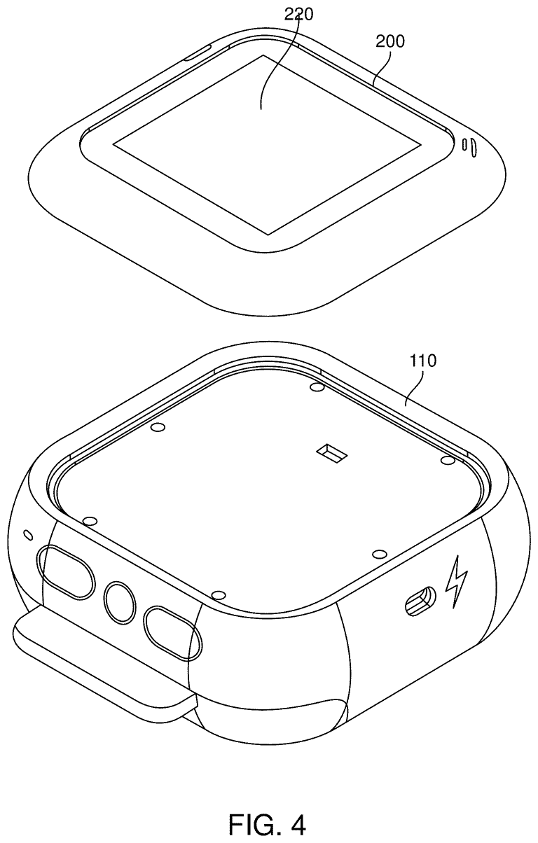

In some embodiments, a paired connection is made between the monitor and the AED—as for example, a Bluetooth, BLE or NFC connection—and the incident notifications are transmitted over the paired connection. In other implementations, the monitor may broadcast beacons (e.g., iBeacons or Eddystone beacons) or advertisements (e.g., BLE advertisements) that contain the incident notifications using processes of the type described in incorporated U.S. Pat. No. 11,452,881 (P016A), and U.S. Pat. No. 11,077,312 (P016B), both of which are incorporated herein by reference. When beacons or advertisements are utilized, the AED is configured to serve as the listener (e.g., a beacon listener that listens for the applicable beacon(s) or a BLE listener that listens for BLE advertisements). When a beacon is used, one of the AED's processors (e.g., the interface unit's processor 210, Bluetooth module 233, or other appropriate controller in the embodiment of FIGS. 4-6 includes a parser configured to decode the beacon and code suitable for directing the AED's response to the beacon.

When the AED receives an incident notification (block 408), it responds by generating an incident alert (block 410). As previously mentioned, the alert may include audible, visual and/or other components that will alert bystanders to the fact that patient has experienced a cardiac arrest and request their assistance in both (a) contacting emergency services (e.g., calling 911 in the U.S.); and (b) responding to the incident—e.g., begin CPR and using the AED to administer a defibrillation shock to the patient as appropriate before emergency services arrive.

The incident notifications sent by the monitor 18 may be transmitted over any of a variety of communications pathways. Since the AED 15 is expected to be located in the general vicinity of the patient, short-range wireless communication protocols such as BLE, Bluetooth, Wi-Fi, Wi-Fi Direct, Near Field Communications (NFC), Zigbee, Z-Wave, Ultra-Wideband (UWB), etc. work well for incident notification messages sent to the AED 15. Incident notifications sent to other nearby devices (e.g., messages sent to nearby Smartphones, tablet computers, smart speakers, etc.) may be sent using the same (or different) short range communication protocols. In some embodiments, the monitor 15, or a base unit associated with the monitor, may additionally or alternatively be arranged to use a longer-range communication system (such as cellular, satellite, Wi-Fi, etc. infrastructures) to send incident messages to one or more of: (i) a pre-designated list of contacts, e.g., family, friends, neighbors, medical personnel, etc.; (ii) a public safety answering point (PSAP)—e.g., a 911 call center in the U.S.; or (iii) a volunteer responder network server.

In some implementations, the monitor broadcasts the incident notification as an advertisement (e.g., as a BLE advertisement) or a beacon (e.g., as an iBeacon, an Eddystone beacon, etc.). There are several benefits of utilizing advertisements or beacons. Notably advertisements and beacons are broadcast (rather than requiring point-to-point connections between the monitor and each nearby recipient device). This enables any/all suitably equipped devices within the beacon's range to receive and appropriately respond to the incident notification (e.g., immediately generate an incident alert) in response to the reception of the beacon, rather than requiring both (a) that each recipient device be known to the monitor; and (b) the establishment of a wireless connection between the monitor and the recipient prior to conveying the incident notification. As will be appreciated by those familiar with beacons, an app installed on a mobile communication device can be designed such that the operating system automatically handles specific tasks triggered by a beacon without requiring that the app even be open at the time. If/when desired, the same can be done on the AED as well—especially when the AED includes a distinct interface unit as described below. Thus, the incident alert can be generated without requiring any corresponding app to be opened prior to generating the incident alert. This is particularly useful for getting incident alerts generated as soon as possible by any/all nearby suitably configured devices—including the AED 15, mobile devices associated with nearby family, friends, etc. Further, since the beacon is broadcast, it can be received by any suitably equipped device, thereby potentially allowing passersby that are unknown to the victim to be notified of, and asked to help respond to, the incident by their own devices.

Any person in the vicinity of the incident that hears or otherwise receives an incident alert can then utilize the AED 15 to try to revive the patient very quickly after the cardiac arrest has occurred. As will be appreciated by those familiar with cardiac arrest, such rapid treatment can significantly improve the patient's survival chances.

In some circumstances, there may not be anyone close enough to the patient at the time of the cardiac arrest to hear the incident alert generated by the AED or to receive the incident notification broadcast by the monitor. Thus, other response pathways are desirable as well. As mentioned previously, some monitors include independent response pathways which can be helpful in many circumstances. In some embodiments, the AED 15 is integrated into an AED inclusive volunteer responder network. Such embodiments enable a variety of additional response pathways. By way of background, the Applicant has developed an AED inclusive responder network that allows Public-Safety Answering Point (PSAP) telecommunicators to notify AEDs and volunteer responders of nearby potential cardiac arrest incidents thereby encouraging volunteers and bystanders to respond to a potential cardiac arrest incident, as well as interact with accepting volunteer responders during their response. Applicant's U.S. Pat. No. 10,580,280 (P014B); U.S. Pat. No. 10,665,078 (P014BC1); U.S. Pat. No. 11,645,899 (P014X3); U.S. Pat. No. 11,210,919 (P021); 11,640,755 (P021X1); and U.S. Pat. No. 11,869,338 (P023) as well as U.S. Patent Publication No. US-2023-0008570-A1 (P025) and others describe a few such systems. Each of these patents and patent applications are incorporated herein by reference.

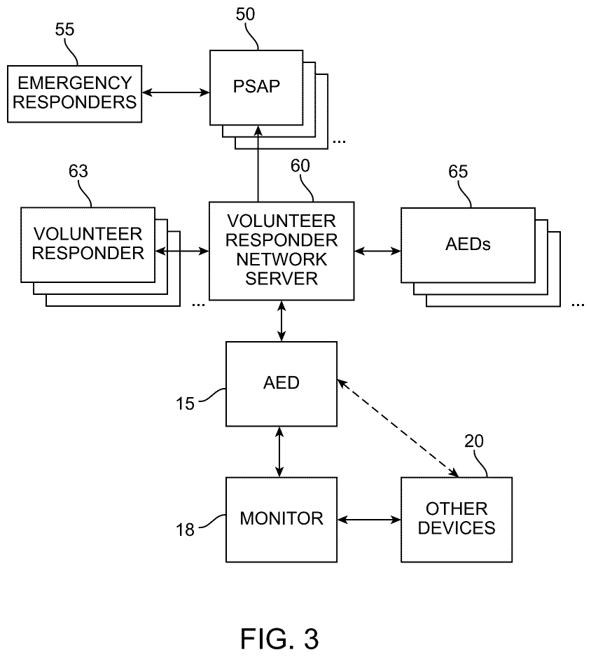

Referring next to FIG. 3, a representative response system is shown that includes emergency dispatch systems (e.g., PSAPs) 50, a volunteer responder network server 60, the AED 15, the monitor 18, emergency responders 55, volunteer responders 63 and other AEDs 65 that are part of the volunteer responder network. In the illustrated embodiment, the volunteer responder network server 60 is provided as an intermediary between the AEDs 65/volunteers 63 and the appropriate PSAP(s) 50. When a telecommunicator wishes to invoke the volunteer responder network, an incident notification is sent to the volunteer responder network server 60, which identifies a set of nearby AEDs and/or volunteers (targets) to notify of the incident and sends appropriate nearby incident notifications to the selected targets 63, 65, 15. In such systems, the volunteer responder network server has the ability to contact the included AEDs 65 and vice versa.

Referring back to FIG. 2, in some embodiments, the AED 15 is configured to send a network incident notification to the volunteer responder network server 60 in response to receiving an incident notification from the monitor 18. Block 430. In some embodiments, the AED simply forwards the received message to the volunteer responder network server, while in others the AED creates its own message or supplements the monitor's notification. In general, the network incident notification includes at least the geolocation of the incident. When the monitor is able to accurately provide its geolocation, the location used in the network incident notification is preferably the location provided by the monitor itself. However, if the monitor does not have geolocation capabilities, the location of the AED may be used in its place since when the monitor 18 can be assumed to be close to the AED 15—e.g., when short-range communication protocols are used for communications between the AED and the monitor. Thus, the AED 15 preferably has the geolocation capabilities—i.e., the ability to determine and report its location. In some embodiments, the network incident notification includes both the monitor location (e.g., a location determined and reported by monitor 18) and the AED location (e.g., a location determined and reported by the AED). Of course, the monitor and AED locations can be determined in other suitable ways. For example, the AED may be configured to estimate/determine the location of the monitor based at least in part on the signal received from the monitor. When the monitor's incident notification is conveyed to the AED by way of an intermediary that is very close to the monitor, intermediary's location (e.g., the location of the intermediary device as determined/provided by the intermediary device such as a cell phone) can be used as an estimate of the monitor's location. The location information also preferably includes location accuracy information such as the Circular Error Probability (CEP) or other measures of the perceived accuracy of the location information as described in U.S. Patent Application No. 63/685,485 (P031P) which is incorporated herein by reference.

The steps taken by volunteer responder network server 60 may vary based on the circumstances and system design preferences. In some circumstances the volunteer responder network may be activated to request assistance from volunteers who are close to the incident. That is, nearby incident notifications are sent to AEDs (block 435) and/or volunteer responders (block 433) that are located near the incident. Volunteer responders could be registered responders, passersby to an AED, etc. The AEDs that are notified may be any AED in relatively close proximity to the incident—e.g., AEDs in the neighborhood. Since the volunteer responder network server is informed of the incident location, it forwards the location information to the selected or accepting responder targets, and a map may be displayed on accepting AEDs or responder devices that provides directions to the incident. More details of a suitable volunteer responder network can be found in the incorporated responder network patents referenced above.

A volunteer response can be particularly useful in circumstances where the AED 15 is not activated relatively quickly, which may suggest that no one is nearby the incident. When activated for an emergency, the AED 15 preferably repeatedly sends status messages to the server indicating its current state (including instruction state), so the volunteer responder network server has good visibility as to if/when the AED is activated, as well as what its current status is, whether a defibrillation shock has been delivered, etc.

In some implementations, the volunteer responder network is activated immediately after the volunteer responder network server receives an incident notification—but is called off if/when the AED is activated indicating that someone at the location is responding to the incident. In other implementations, the volunteer response may not be terminated until EMS is reported to be on scene. This can be particularly helpful because most volunteer responders have at least some training in emergency care and in many cases may be off-duty emergency responders or medical professionals who are particularly well suited for responding to the incident. In other implementations other volunteer response protocols may be followed regarding when to activate the volunteer responder network and/or when to terminate the volunteer responder response.

Since the volunteer responder network server 60 is also in communication with emergency call centers 50, it may also optionally serve as a gateway for automatically notifying the Public Safety Answering Point (PSAP) responsible for the location of the incident. That is, a PSAP incident notification message may be sent from the volunteer responder network server 60 to the responsible PSAP 50 to initiate a professional emergency response-effectively serving the function of a 911 call. Block 437. The PSAP incident notification message 437 also includes the location of the incident so the PSAP can direct emergency responders to the proper location of the incident. Block 439. The location provided in the PSAP incident notification message 437 may take the form of the monitor location (e.g., the monitor location as determined/reported by the monitor) and/or the AED location (e.g., the AED location as determined/reported by the AED).

In some embodiments, the volunteer responder network server also forwards any status messages received from the AED 15 to the PSAP (e.g., to the computer aided dispatch (CAD) system of the PSAP telecommunicator responsible for the incident). This gives the telecommunicator visibility into the scene which can help facilitate telephone/telecommunicator CPR (T-CPR) as described in U.S. Pat. No. 11,210,919 (P021) and U.S. Pat. No. 11,640,755 (P021X1), which are incorporated herein by reference. In some embodiments, communications between the volunteer responder network server 50 and the appropriate PSAP are made by way of an emergency services interface (ESI) (not shown) as described in the incorporated responder network patents.

In the embodiment described above, the network incident notification is sent to the volunteer responder network server 60. In other embodiments, the network incident notification may be sent to an AED management server instead of, or in addition to the volunteer responder network server 60. The management server may then forward the network incident notification to the responder network server, or perform any desired tasks itself. In some embodiments, the AED management server also functions as the volunteer responder network server 60.

Sending the network incident notification to an AED management server 90 has other benefits as well. For example, in some embodiments, the management server is integrated with a management server platform that allows an administrator to define one or more contacts to be notified any time: (i) the AED is activated; and/or (ii) a network incident notification is received based on a potential cardiac arrest detected by a cardiac arrest monitor associated with the AED 15. The contact list may be AED specific or patient specific. For example, a patient specific contact list may include a spouse, children, parents, neighbors, friends, etc. The management server also allows the administrator to set preferences as to how contact notifications should be sent to the designated contacts. This may include text notifications, app-based push notification, other types of instant messaging notifications, e-mail notifications, etc. When the management server receives a network incident notification based on a potential cardiac arrest detected by a particular monitor 18, it immediately sends contact notifications to the people identified in the contact list associated with the associated AED 15.

A benefit of notifying designated contacts is that they may be able to offer assistance or coordinate assistance even if they are not at a location where they can directly hear the alert generated by the AED. For example, a neighbor may not be in range to hear the alert, but if notified may quickly go to the incident location to render aid. In another example, a family member who is not at home who receives a notification of the incident can contact a caregiver or neighbor who is closer to the scene to request their assistance. Of course, there are many other examples as well.

It is noted that the management server's contact notifications are preferably in parallel with any contact notifications that the monitor 18 or a monitor management server 91 may generate independently. Such parallel paths can be quite valuable since the listed contacts for the AED may include individuals that are not identified as contact for the monitor, and vice versa. Also, the AED management server may support certain types of notifications (e.g., text notifications) that are not supported by the monitor contacts, and vice versa.

In the illustrated embodiment, the volunteer responder network server 60 and/or the AED management server is shown as a single box. However, such servers are better thought of as server infrastructures. It should be appreciated that the functionality of the server infrastructure can performed by a single physical and/or virtual server, or any number of physical and/or virtual machines working alone, together or in parallel to perform the various described tasks. As such, the terms volunteer responder network server and/or management server should be understood to include any suitable server infrastructure and may take the form of one or more physical and/or virtual servers that cooperate to handle the described tasks.

In some embodiments, the AED 15 may also be arranged to broadcast its own short range wireless incident notification beacon or advertisement (sometimes referred to herein as repeater incident notifications). Such broadcasts can effectively extend the range of the incident notification sent by monitor thereby increasing the likelihood that the notification will be received by family members or others that are close by the incident. Repeater notifications may simply repeat the monitor's incident notification as received by the AED. In other embodiments, the content of the repeater notifications may also include supplemental information added by the AED, such as the AED's location, whether the transmitting AED received the message directly or indirectly from the monitor, whether the AED has notified the responder network of the incident, etc.

Although particular AED, volunteer responder network and emergency services (PSAP) notification path is illustrated in FIG. 3, it should be appreciated that in other implementations, other notification paths may be desirable—as for example when the sensor of monitor 18 is only designed to communicate with a particular base unit. In such cases, it may be the base unit that acts as the hub for notifying the various response mechanisms. For example, in some implementations, a base unit (which can take the form of the patient's cell phone, a dedicated monitor base station or otherwise) may send the notification received by the AED 15. Alternatively, or additionally, the monitor or an associated base unit may send appropriate incident notifications to one or both of the PSAP 50 or the volunteer responder network server 60.

In some implementations, the monitor/base unit may not be able to directly communicate the incident notification 405 to unit AED 15. This could be due to the AED being out of range of the monitor/base station or due to the capabilities of one or both of the AED or the monitor/base unit. For example, some monitors are designed to only communicate with a single base station and the base station may not be designed to send short range communications of the type that can be received and processed by the AED. In some implementations, the monitor or its base unit may be designed or configured to only communicate with a single monitoring server. Regardless of the reason, in such circumstances, when the base unit is configured to have the ability, it may send an incident notification directly to the volunteer responder network server 60. In other embodiments, the monitor or a base unit may send an incident notification to the monitor management server, which in turns sends an incident notification to the volunteer responder network server. In either case, the volunteer responder network server may then send an incident notification to the AED 15 to thereby cause the AED 15 to generate the incident alert 410. Alternatively, or additionally, the volunteer responder network server can also activate the volunteer responder network by sending nearby incident notifications to nearby AEDs and/or volunteer responders as previously described (435, 433). In this context AED 15 is an AED that the volunteer responder network server knows, or is told, is associated with the monitor. Such information may be conveyed as part of the incident notification sent from the base unit to the volunteer responder network server, or may be an association known to the volunteer responder network server based on information prestored in an AED management database which the correlation being made based on a serial number or other identifying information of the monitor conveyed as part of the incident notification received by the volunteer responder network server. Of course, there may also be circumstances in which there is no AED (or AEDs) associated with a particular monitor and in such circumstances, the volunteer responder network server may simply activate the volunteer responder network (e.g., by sending nearby incident notifications 433, 435).

Applicant's U.S. Patent Publication Nos. 2024-0071197 A1 (P020X1), 2021-0153817 A1 (P020A) and US 2021-0154487 A1 (P020B), each of which are incorporated herein by reference, describe frameworks and mechanism for activating volunteer responder networks from monitors and other devices capable of identifying potential cardiac arrest incidents, as well as integrating virtual assistant systems into an emergency response network. The monitor and/or the AED utilized in the described cardiac arrest detection and treatment kits can utilize the frameworks described therein to activate the volunteer responder network as well.

In still other implementations, the cardiac monitor, or a base station associated therewith may be designed to send an incident notification directly or indirectly to a PSAP which can activate the volunteer responder network as described in the incorporated responder network patents.

In yet further embodiments, the AED 15 may be designed to communicate with PSAPs (e.g., communicate with a PSAP telecommunicator's computer aided-dispatch (CAD) system) by way of an Emergency Services Interface (ESI) or other mechanisms rather than by way of the volunteer responder network server. In such embodiments, an AED 15 receiving a monitor incident notification 405 may initiate or supplement an emergency services response through its connection with the PSAP.

The described cardiac arrest detection monitor/AED integrations have a number of advantages that can help improve a person's chances of surviving out-of-hospital cardiac arrest. When a person is known to be particularly susceptible to cardiac arrest is known it can be advantageous for their physician to prescribe a cardiac arrest detection and treatment kit that includes both a cardiac monitor (wearable, implantable or otherwise). One convenient way to simplify access to the described response framework is to create one or more medical insurance reimbursement code(s) for cardiac arrest monitoring and treatment kits. For example, one code can be utilized for a kit that includes an implantable loop recorder and an accompanying AED, a second code can be utilized for a kit that includes a Holter monitor and an accompanying AED, a third can be used for an ECG/EKG monitor and accompanying monitor, a fourth can be used for a combined fall and heart rate detector capable of identifying potential cardiac arrest events and an accompanying AED. Of course, other additional codes can be utilized for kits that include other specific monitors as may be appropriate.

The described kit and incident notification approaches can be used in conjunction with any defibrillator (including but not limited to AEDs) that is capable of performing the required function. By way of example, and not by limitation, a representative defibrillator system 100 will be described. The illustrated defibrillator utilizes a modular architecture that is well suited for use in automated external defibrillators (including both semi-automated and fully automated defibrillators) although it may also be used in manual defibrillators and hybrid defibrillators that may be used in either automated or manual modes.

The core of the modular defibrillator system 100 is a base defibrillation unit (base unit) 110 as seen in FIG. 4. The illustrated base defibrillation unit 110 is a fully functional AED that is configured such that its functionality can be supplemented by attaching an interface unit 200 to the base unit. The base unit 110 independently functions as an AED both with and without the attached interface unit 200. In this embodiment, interface unit 200 includes one or more processors, a touch sensitive display screen 220, a communications unit and preferably its own power storage unit (e.g., battery). The touch sensitive display facilitates user interactions with the interface unit and, as appropriate, indirect interactions with the base unit. The communications unit facilitates communications with external systems and/or devices using a variety of different communications technologies and protocols, as for example, Wi-Fi communications, cellular communications, satellite communication and short-range wireless communications technologies (e.g., Bluetooth, Near Field Communication (NFC), etc.) By way of example, U.S. Pat. No. 10,773,091 (P006E), U.S. Pat. No. 10,737,105 (P006A), U.S. Pat. No. 11,452,881 (P016A), U.S. Pat. No. 11,077,312 (P016B) and U.S. patent application Ser. No. 17/007,838 (P019B), each of which is incorporated herein by reference, describe details of a variety of such modular defibrillator architectures.

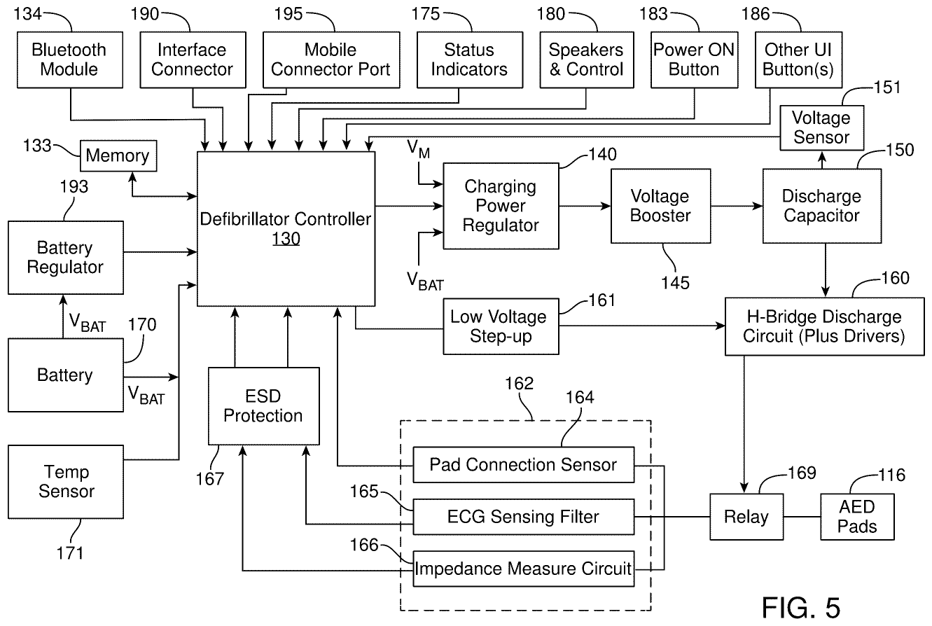

FIG. 5 is a block diagram illustrating one representative electronics control architecture and associated components suitable for use in the base defibrillator unit 110. In the illustrated embodiment, the electronic components include a defibrillator controller 130, memory 133, a wireless communications module in the form of Bluetooth module 134, a charging power regulator 140, a voltage booster 145 (which may have multiple stages), a high voltage capacitor 150 for temporarily storing sufficient electrical energy suitable to provide a defibrillation shock, discharge control circuitry 1160, pad related sensing circuitry 162 and relays 169, power storage unit 170, battery regulator 193, status indicator(s) 175, speaker(s) 180 and one or more electrical connectors (e.g., interface connector 190, mobile connector port 195, charger connector (not shown), etc.). The charging power regulator 140 and voltage booster 145 which cooperate to control the charging of the shock discharge capacitor 150 are sometimes referred to herein as a charging circuit.

The defibrillator controller 130 is configured to control the operation of the base defibrillator unit and to direct communications with external devices, as appropriate. In some embodiments, the defibrillator controller includes a processor arranged to execute software (some or all of which may take the form of firmware) having programmed instructions for controlling the operation of the base unit, directing interactions with a user and communications with external components. The software may be installed on the memory 133. Although the singular term memory is often used herein, it should be appreciated that the memory may be divided into multiple different parts which take any suitable form or combination of forms (e.g., various types of RAM, ROM, PROM, EEPROM, etc.) Unless the context suggests otherwise, references to “memory” herein are intended to cover all suitable forms and combinations of physical memory. Similarly, although the singular term “processor” is often used herein, it should be appreciated that any appropriate number of processors and/or processing cores can be utilized and unless the context suggests otherwise, references to “processor” herein are intended to cover processing units composed of one or more physical processors or processing cores.

The base defibrillator unit 110 may optionally be configured so that it is capable of drawing power from certain other available power sources beyond power storage unit 170 to expedite the charging of shock discharge capacitor 150. The charging power regulator 140 is configured to manage the current draws that supply the voltage booster, regardless of where that power may originate from. For example, in some embodiments, supplemental power may be supplied from a mobile device coupled to mobile connector port 195 or from a portable charger/supplemental battery pack coupled to charger connector 197.

The voltage booster 145 is arranged to boost the voltage from the operational voltage of power storage unit 170 to the desired operational voltage of the discharge capacitor 150, which in the described embodiment may be on the order of approximately 1400V-2000V (although the defibrillator may be designed to attain any desired voltage). In some embodiments, the boost is accomplished in a single stage, whereas in other embodiments, a multistage boost converter is used. A few representative boost converters are described in the incorporated U.S. Pat. No. 10,029,109. By way of example, in some embodiments, a flyback converter, as for example, a valley switching flyback converter may be used as the voltage booster 145—although it should be appreciated that in other embodiments, a wide variety of other types of voltage boosters can be used.

A voltage sensor 151 is provided to read the voltage of the capacitor 150. The voltage sensor 151 may take the form of a voltage divider or any other suitable form. This capacitor voltage reading is utilized to determine when the shock discharge capacitor 150 is charged suitably for use. The sensed voltage is provided to controller 130 which determines when the capacitor 150 is charged sufficiently to deliver a defibrillation shock. The capacitor 150 can be charged to any desired level. This can be useful because different defibrillation protocols advise different voltage and/or energy level shocks for different conditions. Furthermore, if the initial shock is not sufficient to restart a normal cardiac rhythm, some recommended treatment protocols call for the use of progressively higher energy impulses in subsequently administered shocks (up to a point).

The discharge circuitry 160 may take a wide variety of different forms. In some embodiments, the discharge circuitry 160 includes an H-bridge along with the drivers that drive the H-bridge switches. The drivers are directed by defibrillator controller 130. The H-bridge outputs a biphasic (or other multi-phasic) shock to patient electrode pads 116 through relays 169. The relays 169 are configured to switch between an ECG detection mode in which the patient electrode pads 116 are coupled to the pad related sensing circuitry 162, and a shock delivery mode in which the patient electrode pads 116 are connected to H-Bridge to facilitate delivery of a defibrillation shock to the patient. Although specific components are described, it should be appreciated that their respective functionalities may be provided by a variety of other circuits.

The pad related sensing circuitry 162 may include a variety of different functions. By way of example, this may optionally include a pad connection sensor 164, ECG sensing/filtering circuitry 165 and impedance measurement chip/block 166. The pad connection sensor is arranged to detect the pads are actually connected to (plugged into) the base defibrillator unit 110. The ECG sensing/filtering circuitry 165 senses electrical activity of the patient's heart when the pads are attached to a patient. The filtered signal is then passed to defibrillator controller 130 for analysis to determine whether the detected cardiac rhythm indicates a condition that is a candidate to be treated by the administration of an electrical shock (i.e., whether the rhythm is a shockable rhythm) and the nature of the recommended shock. When a shockable rhythm is detected, the controller 130 directs the user appropriately and controls the shock delivery by directing the H-bridge drivers appropriately.

In some embodiments, the power storage unit 170 takes the form of one or more batteries such as rechargeable Lithium based batteries including Lithium-ion and other Lithium based chemistries, although other power storage devices such as one or more supercapacitors, ultracapacitors, etc. and/or other battery chemistries and/or combinations thereof may be used as deemed appropriate for any particular application. The power storage unit 170 is preferably rechargeable and may be recharged via any of a variety of charging mechanisms. In some embodiments, the power storage unit 170 takes the form of a rechargeable battery. For convenience and simplicity, in much of the description below, we refer to the power storage unit 170 as a rechargeable battery. However, it should be appreciated that other types of power storage devices can readily be substituted for the battery. Also, the singular term “battery” is often used, and it should be appreciated that the battery may be a unit composed of a single battery or a plurality of individual batteries and/or may comprise one or more other power storage components and/or combinations of different power storage units.

In some embodiments, the base defibrillator unit 110 is capable of drawing power from other available power sources for the purpose of one or both of (a) expediting the charging of shock discharge capacitor 150 and (b) recharging the power storage unit 170. In some embodiments, the battery can be recharged using one or more of the externally accessible connector ports 195, a dedicated charging station, a supplemental battery pack (portable charger), an interface unit 200, etc. as will be described in more detail below. When wireless charging is supported, the base defibrillator unit may include a wireless charging module 174 configured to facilitate inductive charging of the power storage unit 170 (e.g., using an inductive charging station 294, or other devices that support inductive charging, as for example an inductively charging battery pack, a cell phone with inductive charging capabilities, etc.).

The base unit also includes a number of software or firmware control algorithms installed in memory 133 and executable on the defibrillator controller. The control algorithms have programmed instructions suitable for controlling operation of the base unit and for coordinating the described broadcasts, as well as any point-to-point communications between the base unit 110 and the interface unit 200, connected devices, and/or any other attached or connected (wirelessly or wired) devices. These control routines include (but are not limited to): communication control algorithms, heart rhythm classification algorithms suitable for identifying shockable rhythms; capacitor charge management algorithms for managing the charging of the discharge capacitor; capacitor discharge management algorithms for managing the delivery of a shock as necessary; user interface management algorithms for managing the user instructions given by the defibrillator and/or any connected user interface devices (e.g. interface unit 200, mobile communication device 105) during an emergency; battery charge control algorithms for managing the charging of power storage unit 170; testing and reporting algorithms for managing and reporting self-testing of the base unit; software update control algorithms and verification files that facilitate software updates and the verification of the same.

In many installations, an AED will be expected to be stored for long periods of time without being plugged into power and therefore relying solely on battery power. Accordingly, it is important to minimize the power drain as much as possible during this time. In some embodiments, the defibrillator controller is configured to shut down power to all electrical components (including itself) except for (a) a real time clock (not shown), (b) the Bluetooth module 134, and (c) a power controller (not shown) when the AED is in the standby (resting) mode. In some embodiments, the clock and/or the power controller are integrated into the Bluetooth module 134 or an I/O adaptor board that includes the Bluetooth module. The clock is configured to periodically send a status check alarm to the power controller which wakes the power controller up from a sleep mode when the power controller is separate from the Bluetooth module. In response to the status check alarm, the power controller restores power to the entire system. Once power is restored, the defibrillator controller 130 performs a status check and instructs the Bluetooth module 134 to update the standby status message 150 as appropriate. After the status check is performed, the defibrillator controller will again shut down power to all electrical components except the clock, the Bluetooth module 134 and the power controller. The status check alarms can be generated at any desired intervals, as for example, once each day.

With the described power management scheme, the Bluetooth module 134 can continue to broadcast standby status messages even when the AED is powered down.

If the Bluetooth Module 134 makes a connection while the AED is in the standby mode, it sends a message to the power controller, which in turn, powers on the system, thereby allowing the defibrillator controller or other suitable component to communicate with the connecting device. Thus, the AED can effectively be woken up via a Bluetooth connection request. Of course, the power controller is also configured to power the system when a user presses the AED's “ON” button or otherwise activates the AED.

In other embodiments, one or more of the electrical components of the AED, such as the defibrillator controller 130, can be placed in a “sleep” mode rather than being turned off when the AED is in the standby mode. However, a significant advantage of actually turning the defibrillator controller and the bulk of the AED's electrical components off as opposed to placing them in a sleep mode is that it eliminates the power draw associated with the sleep mode, thereby potentially extending the AED's shelf life. It should be appreciated that the power controller/power down approach can be also used with AEDs that don't incorporate a Bluetooth module and/or support the low energy broadcasts described herein.

In some embodiments, a temperature sensor 171 is provided within the defibrillator itself for detecting the internal temperatures of the AED (as opposed to an environmental temperature), which is then used in the temperatures used to trigger the temperature fault notification and clearance messages. Preferable the temperature sensor is positioned adjacent one of the more temperature sensitive components such as battery 170 so that the reported temperature is directly related to the internal temperature of the AED near the temperature sensitive component.

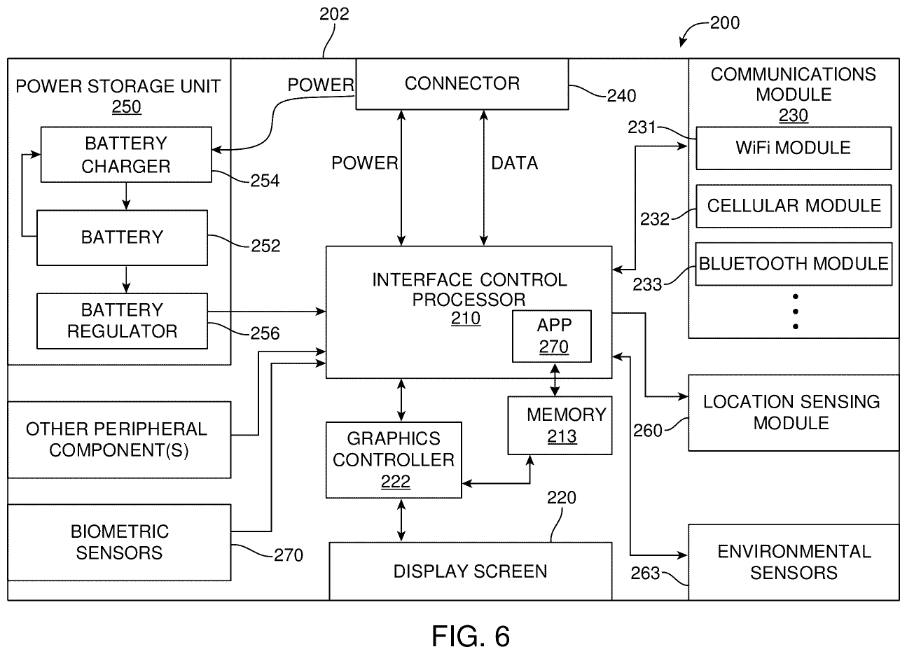

FIG. 6 illustrates some of the electrical components of a representative interface unit 200. In the illustrated embodiment, the interface unit 200 includes an interface controller (processor) 210, memory 213, a display screen 220, a communications module 230, an electrical connector 240, an interface unit power storage unit 250, and a location sensing module 260, all of which may be housed within the interface unit housing 202. The interface unit may also have software or firmware (such as an app 270) installed or installable in memory 213 having programmed instructions suitable for controlling operation of the interface unit and for coordinating communications between the interface unit 200 and the base defibrillation unit 110 and/or remote devices.

The processor 210 controls operation of the interface unit and coordinates communications with both the base unit 110 and remote devices such as a central server (as will be described in more detail below). In some embodiments, the processor 210 is arranged to execute a defibrillator app 270 or other software that can be used both during use of the defibrillator system 100 during a cardiac arrest incident and to facilitate non-emergency monitoring or/or use of the defibrillator system 100. Similar to the base unit processor 130 discussed above, unless the context suggests otherwise, the processor 210 may take the form of a single processor, multiple processors, multiple processing cores and other processing unit configurations.

The display screen 220 is a touch sensitive screen suitable for displaying text, graphics and/or video under the direction of the processor 210 to assist both during both emergency situations and at other times. The touch sensitive screen is configured to receive inputs based on a graphical user interface displayed thereon. In some embodiments an optional graphics controller 222 may be provided to facilitate communications between the interface control processor 210 and the display screen 220. In other embodiments, functionalities of the graphics controller may be part of the processor 210.

The communication module 230 is provided to facilitate communications with remotely located devices such as the central server. The communications module 230 may be configured to utilize any suitable communications technology or combination of communication technologies including one or more of cellular communications, Wi-Fi, satellite communications, Bluetooth, BLE (Bluetooth Low Energy), NFC (Near Field Communications), Zigbee communications, DSRC (Dedicated Short-Range Communications) or any other now existing or later developed communications channels using any suitable communication protocol. By way of example, in the illustrated embodiment, the communications module 230 includes Wi-Fi, cellular and Bluetooth modules 231, 232 and 233 that facilitate Wi-Fi, cellular and Bluetooth communications respectively. Preferably the BLE module 233 and interface control processor 210 in the interface unit 200 is set up as a listener (rather than the Bluetooth module 134 in the base defibrillator unit 110) to receive communications from the cardiac monitor 18 and the various steps described as being performed by the AED with respect to FIG. 2 are performed by the interface unit 200. E.g., the listener functionality, the transmission of notifications to the volunteer responder network server, nearby AEDs or volunteers, etc. are preferably performed by the interface unit. Such an approach is advantageous from a security standpoint and also helps prevent the base unit's battery 170 from being unnecessarily depleted.

The electrical connector 240 is configured to mate with interface connector 190 on the base defibrillator unit 110. The connectors 190 and 240 are configured to facilitate communications between the defibrillator controller 130 and the interface unit's processor 210. The connectors 190 and 240 are also preferably arranged to supply power from the interface unit 200 to the base unit 110 as will be described in more detail below. In some embodiments, power will only be provided in one direction—i.e., from the interface unit 200 to the base unit 110 and not in the reverse direction during operation. A good reason for this approach is that the defibrillator is the most important component from a safety standpoint, and it is often undesirable to draw power from the base unit to power other devices (including the interface unit 200) in a manner that could reduce the energy available to charge the discharge capacitor in the event of an emergency. However, in some embodiments, the power supply may be bi-directional (at least in some circumstance) if desired—as for example if the base unit is not in use, is fully charged and plugged into an external charging power supply, etc.; or if the power passed to the interface unit is not coming from the base unit's internal battery (e.g., it is coming from a charger, a mobile communication device, or other device connected or attached to the base unit), etc.

The connectors 190 and 240 can take a variety of forms. They can be connectors with accompanying transceivers configured to handle processor level communications (such as UART, SPI, or I2C transceivers), with additional pins for power delivery (Power+GND), and connection verification (i.e. a pin that detects when there is a connection between the interface unit and the Base AED and triggers an interrupt on the Base AED signifying that there is not a unit connected). They can also be more standardized connectors such as USB connectors.

The interface power storage unit 250 provides power to operate the interface unit 210. In many embodiments, the power storage unit takes the form of a battery 252 with associated control components, although again a variety of other power storage technologies such as supercapacitors, ultracapacitors, etc. may be used in other embodiments. The associated control components may include components such as a battery charger and maintainer 254, which may include various safety monitors, and battery regulator 256. Preferably, the power storage unit 250 is rechargeable, although that is not a requirement. In some embodiments it may be desirable to utilize replaceable batteries (rechargeable or not) so that the batteries in the power storage unit 250 can be replaced when they near the end of their useful life. In some embodiments, the power storage unit 250 may also be arranged to supply supplemental power to the base unit 110. Depending on the structure and/or state of the base unit, the supplemental power can be used to help charge the discharge capacitor 150 during use; to power or provide supplemental power for the defibrillator electronics and/or to charge the base defibrillator unit's power storage unit 1170. In other embodiments, a supplemental battery within the interface unit (not shown) may be used to provide the supplemental power for the base unit rather than the power storage unit 250.

The location sensing module may incorporate a variety of technologies including Global Navigation Satellite Systems (GNSS) (e.g., GPS), Wi-Fi positioning, cellular triangulation, assisted GPS, Bluetooth Beacons, Near-Field Communications (NFC) and/or other location determining technologies. When requested, the interface unit can report its current location based on the location sensing technology that is believed to have the best accuracy under the then-present circumstances.

The interface unit may also optionally include various environmental sensors 263 and other peripheral components 266. When desired, the interface unit may include any of a wide variety of different types of sensors and peripheral components. For example, in selected embodiments, the interface unit may include one or more accelerometers and/or gyroscopes, a temperature sensor, a humidity sensor, a time of day or any other desired sensors or components.

The interface unit 200 is preferably configured to securely mechanically attach to the base unit 110. Typically, the interface unit is detachable such that it may be separated from the base unit if desired—although in other embodiments, the attachment may be more permanent in nature. The specific mechanical attachment utilized may vary widely in accordance with the needs of any particular embodiment. In some embodiments, press or form fitting attachment structures are used, while in others, latch and catch mechanisms, snap fit structures, etc. are utilized alone or in combination to releasably attach the interface unit to the base. However, it should be appreciated that a wide variety of other structures can be used in other embodiments. In some embodiments, the interface unit includes an attachment sensor (not shown) that senses when the interface unit is attached to a base unit.

In some embodiments, the interface unit may also include one or more biometric sensors 270. The biometric sensors may vary based on the needs of any particular defibrillator. Some of the biometric sensors may be suitable for use in detecting or evaluating CPR performed during emergency use of the defibrillator. Other biometrics may be useful in more general health management applications. For example, in some embodiments, the biometric sensors may include one or more of a pulse or heart rhythm sensor, a blood pressure sensor, a glucose monitor, a pulse oximeter, an ECG monitor, a sleep tracker, a thermometer, etc.

A benefit of the described modular defibrillator architecture is that the interface unit can be (and preferably is) designed to provide robust connectivity, effectively making the defibrillator a highly connected device. The relatively large touch sensitive display screen provides an interface that can be easily used by users of most any age, and the dedicated interface unit processor(s) and corresponding memory allow the interface unit to be programmed to provide a number of functionalities that are not available in defibrillators that are commercially available today.

It should be appreciated that the described modular architecture helps overcome a number of practical challenges that have hindered widespread adoption of defibrillator connectivity. One particular challenge relates to security. If a defibrillator has the ability to bidirectionally communicate with the external systems through the Internet or other public networks, such connectivity introduces security risks that wouldn't otherwise exist. This can also lead to regulatory challenges. For example, a practical challenge that is faced when incorporating a communications unit directly into the AED is that, in general, AEDS are highly regulated medical devices. For example, in the United States, new AEDs are Class III Medical Devices that require premarket approval (PMA) from the U.S. Food and Drug Administration (FDA) prior to sale in the United States—which is the most stringent type of medical device marketing application required by the FDA. The PMA approval process is quite rigorous and can take a significant amount of time. When the communications unit is an integral part of the AED it is subject to the full PMA review process which means that anytime there is a desire to update any of the communication unit's software, such updates can subject to extensive review process. In practice, the software used in many communications technologies tends to be updated or patched on a fairly regular basis. Such updates may be designed to patch newly discovered security vulnerabilities, eliminate bugs, facilitate compatibility with additional devices or protocols, add new functionalities, and/or for a variety of different purposes. Unfortunately, such regular updates are fairly incompatible with the PMA review process which tends to be protracted and expensive.

Given that the interface unit is a connected device that has a powerful processor (or processors) and a number of familiar I/O components including, for example, a touch sensitive display screen, Wi-Fi, cellular and other wireless communications capabilities, a speaker, a microphone and optionally a camera, the interface unit can be programmed to provide a number of useful functionalities without impacting the functionality of the base defibrillator unit in any way.

In many of the embodiments described above, the cardiac monitor 18 is configured to communicate directly with the AED 15 or via a local intermediary—e.g., a base unit or Smartphone in the vicinity of both the monitor and the AED. However, in other implementations, the monitor may be configured to communicate with a remotely located server (e.g., a monitor management server or an AED management server) which in turn communicates directly or indirectly with the AED.

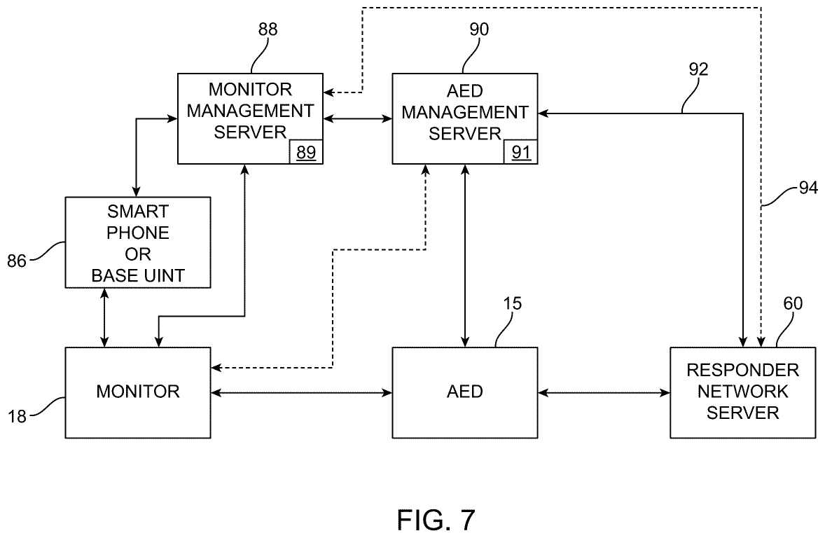

Referring next to FIG. 7, alternative AED notification pathways that are server based will be described. Presently, many cardiac monitors 18 are configured to communicate with a remotely located monitor management server 88 either directly or indirectly via a base station or smart phone 86. Such monitors may also report conditions of concern (including, but not necessarily limited to cardiac arrest incidents) to the monitor management server 88 in real-time when the event occurs. Since the monitor management server 88 knows of the event in real time, it may serve as a conduit for alerting an AED 15 associated with the monitor and/or activating a volunteer responder network.

In some such implementations, when the monitor detects a potential cardiac arrest, a monitor incident notification is sent from the monitor 18 or an associated base unit 86 (which may be a smartphone) to the monitor management server 88. The monitor incident notification includes an identifier that uniquely identifies the monitor and further indicates that the monitored patient may be experiencing a cardiac arrest. The monitor management server 88 has a database 89 that indicates whether the monitor has an associated AED 15. Upon receiving the monitor incident notification, the monitor management server 88 checks the database 89 to determine whether the reporting monitor has an associated AED. If so, the monitor management server sends an incident notification to the AED management server 90, which has the ability to communicate with the associated AED. In other embodiments, the monitor management server is configured to send a network incident notification to the AED management server any time a potential cardiac arrest is detected. The AED management server 90 has a management database 91 that identifies the specific AED(s) associated with specific monitors. Thus, upon receipt of the incident notification from the monitor management server 88 that includes a monitor identifier, the AED management server looks up the AED(s) that are associated with the identified monitor in the AED management database. Once the associated AED(s) has/have been identified, the AED management server 90 sends an incident notification to the associated AED(s).

When the associated AED 15 receives the incident notification, it generates an audio and/or visual incident alert that hopefully draws the attention of people nearby the AED and encourages them to act to treat the cardiac arrest victim using the AED 15.

The specific content of the various incident notifications in the described server-based AED notification path may vary based on the capabilities of the monitor and design preferences. In various embodiments, the incident notifications set to the server may include any of the content described above with respect to incident notifications sent from the monitor to an AED. In some embodiments, either the incident message itself or separate communications may convey detected ECG segments of interest to the monitor management server 88. This could be simply the ECG segments used in a classification of the potential cardiac arrest, or more extended segments. In some embodiments, the monitor may not make the cardiac arrest diagnosis itself, but rather simply report detected anomalies and the corresponding ECG segments to either the base unit 86 or the monitor management server 88 which in-turn, makes the cardiac arrest diagnosis, thereby triggering the remainder of the server-based AED notification process.

The incident notifications sent from the monitor management server 88 to the AED management server 90 may include any/all of the information contained in the monitor incident notification plus any additional information that may be relevant to the AED management server 90. Typically, the monitor management server 88 will have additional information about the victim that may be helpful to the response. This can include the patient/victim's name, address, demographic information about the victim (such as age, gender, etc.), and relevant medical history information (e.g., the patient has a condition involving a low ejection fraction, etc.). When available, the monitor management server's incident notification sent to the AED management server 90 can include such additional information. When desired, some or all of the relevant patient specific information can be sent in messages that follow the initial incident notification sent to the AED management server.

The incident notification sent from the AED management server to the AED preferably includes at least the geolocation of the victim so that the AED can display a map that shows a responder responding to the incident alert where the victim is located and, when appropriate, how to navigate to the incident. The incident notification may also include notes that are relevant to the incident that may be helpful to the responder. For example, the notes may provide a pre-set description of the patient, or the patient's expected location. For example, in the context of an assisted living situation, the notes may say something like Mr. Smith in apartment 3B is experiencing a cardiac arrest. It can also include notes about the patient such as their age, gender, allergies, etc. that may be helpful for potential responders.

When a patient has multiple expected locations, (e.g., a home and vacation home or an office) the notes can be customized for the location of the incident. This is possible because the incident notification generated by the monitor includes the patient location. Thus, the management server can check the incident location against one or more designated expected locations. For example, if the incident location matches a primary home location, direction information relevant to that home may be provided in the notes, whereas if the incident location matches an alternate know location (e.g., a vacation home, an office, etc.) direction information relevant to the actual incident location may be provided.

In some circumstances, the monitor server-based notification path may also initiate a volunteer responder network by sending a responder network activation request to a responder network server 60. The responder network activation request can be sent to responder network server 60 by either monitor management server 88 (as represented by notification pathway 94), or by the AED management server (as represented by notification pathway 92). The responder network activation request includes the geo-location of the incident, preferably together with any other relevant information that is available to the requesting server (e.g., the time that the potential cardiac arrest was detected, patient information, etc.). When the responder network server 60 receives a responder network activation request, it identifies nearby volunteer responders and/or AEDs and sends nearby incident notifications to such devices. Suitable responder networks are described, for example, is U.S. Pat. No. 10,580,280 (P014B); U.S. Pat. No. 10,665,078 (P014BC1); U.S. Pat. No. 11,645,899 (P014X3); and U.S. Patent Publication No. 2024-0071197 A1 (P020X1), each of which are incorporated herein by reference.

One advantage of the server-based AED notification approach is that many cardiac monitors are already designed to communicate incident notifications to an associated monitor management server. Such monitors can thus easily be effectively integrated with an associated AED and/or effectively enable volunteer responder network responses by making the described monitor management server integrations with the AED management server and/or the responder network without requiring any modification of the monitors themselves.

Another advantage of the server-based AED notification approach is that it potentially extends the range at which the associated AED may be notified of an incident. Specifically, direct monitor to AED communications would typically be via a known short range wireless communications protocol such as BLE, NFC, Wi-Fi Direct, etc. Since the range of such systems is relatively limited, there is a risk that the AED may not be within range of the monitor even if they are both within the same household due to factors such as the distance between the devices and intervening structures (e.g., walls, appliances, etc.).

Conversely, there may be times when direct communications between the monitor and the AED may be quite effective but longer range communications between either the monitor and the monitor management server, or the AED and the AED management server may be impeded for some reason (e.g., a Wi-Fi network relied on for such communications may be down or one of the devices may be in a location that lacks good cellular or Wi-Fi network coverage when such networks are relied on for longer range communications). Therefore, there are advantages to facilitating both the direct and server-based AED notification pathways. In such systems, the AED management server and/or the AED may be configured to recognize that more than one notification has been received for a particular event. This is easily done by each relevant node (e.g., the AED and the AED management server) checking the incident identifier in any received message to determine whether it has previously received a notification of that incident and responding accordingly.

Yet another advantage of the server based AED notification approach is that in circumstances where there is/are no AED(s) associated with the cardiac arrest monitor, or the associated AEDs are not nearby when the cardiac arrest occurs, the server (which may be the AED management server or the monitor management server) may activate the volunteer responder network to encourage 3^{rd} party volunteers to respond to the incident. Of course, the volunteer responder network may be activated even when there are nearby associated AEDs.

Although only a few embodiments of the invention have been described in detail, it should be appreciated that the invention may be implemented in many other forms without departing from the spirit or scope of the invention. For example, although much of the description focuses on circumstances in which the AED 15 is associated in some way with a particular cardiac monitor or patient, the described AED functionality can be integrated into any AED so that the patient can benefit from any AED that may happen to be located nearby when a monitor detects that the patient is in cardiac arrest.

In some of the described response pathways, the volunteer responder network server 60 activates a volunteer responder network by sending nearby incident notifications to nearby volunteer responders 433 and/or nearby AEDs 435. In some embodiments, the volunteer responder may have or obtain notes associated with the monitor 18 or the patient. Such notes can be received as part of the incident notification, received in separate messages, or prestored in a management database. Such notes can be provided to volunteer responders as appropriate. By way of example, the notes can indicate how to access the patient's location, where the patient's AED is located or stored, etc. For example, a note could indicate that the patient is in cardiac arrest and that a functional AED is located at a particular location. This can be a present message along the lines of an AED is located in the kitchen, or I always keep my AED in my black go-bag. In some implementations, the volunteer responder network server can ping the AED 15 to request that it report its current location if hasn't already received the current location as part of an incident notification. The reported location can then be conveyed to volunteer responders as appropriate inform them of the AED's presence. Finding the AED can be further facilitated by having the AED 15 continue to generate loud and/or highly visible incident alerts until the AED is activated or the incident is terminated so that the AED is more readily noticed when a volunteer responder arrives on scene.

In many circumstances one or both of the monitor or the AED will have an associated App that pairs with the monitor or AED. Preferably the patient will have such apps installed on their cell phone and/or other mobile communications devices. Any such app preferably has a contact list of emergency contacts to be notified of the fact that the patient is in cardiac arrest. If/when the patient's mobile device receives an incident notification from the monitor 18, the monitor or AED app will automatically open and send push notifications (e.g., text messages) to each of the listed contacts. Messages sent to the emergency contacts can also include notes—for example, a text sent to family members may say something like: “Bob is in cardiac arrest, get the AED from the kitchen cabinet and begin emergency treatment.” Of course, the notes can convey any information deemed to likely be helpful to people responding to the incident.

When a person in the patient's contact list also has the app installed on their mobile device, a prompt can be generated on their device asking whether the person is actually responding. They can also be asked whether they are responding with or without an AED. If they are responding with an AED, then there may be no need to activate the volunteer responder network, or it may be appropriate to call off the volunteer responder response. Alternatively, if they are responding without an AED, it may still be desirable to request volunteer or bystander assistance in bringing an AED to the scene of the incident.

In the discussion above, frequent references are made to cardiac arrest monitors and cardiac monitors. In general, a cardiac arrest monitor or a cardiac monitor will include both a sensor and a communications unit that is capable of communicating with external and remotely located devices. In some circumstances, the communications unit capable of communicating with external and remotely located devices may be part of a base unit that communicates with the sensor via a wireless or wired connection. Therefore, unless the context suggests otherwise, the terms cardiac arrest monitor and cardiac monitor as used herein should be understood to include embodiments in which a sensor and a base unit that provides the required external connectivity are embodied in separate components.

Although the invention has been primarily discussed in the context of cardiac monitors used as the cardiac arrest monitor, it should be appreciated that in other embodiments, other types of monitors that are capable of detecting potential cardiac arrest incidents may be used in place of, or as additions to a cardiac monitor. For example, as described in the incorporated U.S. Patent Publication No. 2024-0071197 A1 (P020X1), a variety of different types of monitors can be configured and used to detect potential cardiac arrest incidents. These include various devices capable of detecting falls, devices capable of detecting agonal breathing, heart rate and/or pulse sensors, respiration sensors, motion sensors, etc. and combinations thereof that are configured to identify and report potential cardiac arrest incidents. For example, a “help I can't get up” 911 alert device, a security or home camera, a wearable, watch, or environmental motion detector that is able to detect a patient fall and/or subsequent unresponsiveness can be used to detect potential cardiac arrest. In different embodiments, these types of devices may be used as the cardiac arrest monitor or combined with a cardiac monitor to constitute the cardiac arrest monitor.

In some implementations described above, beacons or advertisements are used to convey incident notifications, with BLE advertisements/beacons being used as a primary example. It should be appreciated that the described beacons and/or advertisements can be used in conjunction with a variety of other short range communication technologies as well, as for example, Wi-Fi Direct, Wi-Fi Aware, Zigbee, NFC and others. Therefore, the present embodiments should be considered illustrative and not restrictive and the invention is not to be limited to the details given herein, but may be modified within the scope and equivalents of the appended claims.

What is claimed is:

1. An automated external defibrillator (AED) comprising:
a shock delivery circuit capable of delivering a defibrillation shock;
a receiver configured to wirelessly receive a first incident notification transmitted by a monitor indicating that the monitor has detected a potential cardiac arrest in a patient monitored by the monitor; and
wherein the AED is configured to, in response to reception of the first incident notification, (i) generate a human perceptible incident alert to inform individuals that are nearby both the cardiac monitor and the AED of the potential cardiac arrest to thereby encourage such individuals to respond to the potential cardiac arrest incident using the AED, and (ii) send a second incident notification to a remotely located server to thereby inform the remotely located server of the potential cardiac arrest incident, the second incident notification being sent in real time relative to the reception of the first incident notification.

2. An automated external defibrillator as recited in claim 1 wherein the remotely located server is one selected from the group consisting of a volunteer responder network server and an AED management server.

3. An automated external defibrillator as recited in claim 1 wherein the receiver that receives the first incident notification is a Bluetooth Low Energy (BLE) receiver.

4. An automated external defibrillator as recited in claim 3 wherein the first incident notification is a broadcast selected from the group consisting of a beacon and a BLE advertisement.

5. An automated external defibrillator as recited in claim 1 further comprising a speaker and wherein the human perceptible incident alert is or includes an auditory alert generated by the speaker.

6. An automated external defibrillator as recited in claim 1 further comprising a display screen and wherein the human perceptible incident alert is or includes a visual alert displayed on the display screen.

7. An automated external defibrillator as recited in claim 3 further comprising a BLE transmitter and wherein the AED is further configured to serve as a repeater that rebroadcasts the first incident notification using the BLE transmitter.

8. An automated external defibrillator as recited in claim 1 wherein the monitor is a cardiac monitor.

9. A kit that includes the automated external defibrillator as recited in claim 1 and the monitor.

10. A cardiac arrest detection and treatment kit comprising:
a monitor having a first transmitter, the monitor configured to detect a potential cardiac arrest in a patient monitored by the monitor, and to cause the first transmitter to wirelessly transmit a first incident notification in response to the detection of the potential cardiac arrest; and
an automated external defibrillator (AED) separate from and associated with the monitor, the AED including a first communication unit configured to communicate with a remotely located server and a second communications unit configured to at least one of send or receive short-range wireless communications, the AED configured to wirelessly receive at least one of the first incident notification transmitted by the cardiac monitor or a second incident notification received from the remotely located server that is directly or indirectly triggered by the first incident notification, the AED being further configured to,
(i) in response to reception of the first or second incident notification, generate a human perceptible incident alert to inform individuals that are nearby the AED associated with the cardiac monitor of the potential cardiac arrest to thereby encourage such individuals to respond to the potential cardiac arrest incident using the AED, and
(ii) communicate with the remotely located server regarding the potential cardiac arrest incident.

11. A cardiac arrest detection and treatment kit as recited in claim 10 wherein the monitor is a cardiac monitor.

12. A cardiac arrest detection and treatment kit as recited in claim 10 wherein the cardiac monitor is selected from the group consisting of a Holter monitor, an implantable cardiac monitor (ICM) and an ECG/EKG monitor.

13. A cardiac arrest detection and treatment kit as recited in claim 10 wherein the monitor includes a fall detector.

14. A cardiac arrest detection and treatment kit as recited in claim 10 wherein:
the remotely located server is an AED management server;
the first incident notification is sent directly or indirectly from the cardiac monitor to a monitor management server, which sends a third incident notification directly or indirectly to the AED management server; and
the AED management server directly or indirectly sends the second incident notification to the AED.

15. A cardiac arrest detection and treatment kit as recited in claim 10 wherein the AED is configured to receive and act upon both first incident notifications and second incident notifications.

16. A cardiac arrest detection and treatment kit as recited in claim 10 wherein the AED is configured to be capable of acting upon one of the first incident notification and the second incident notification and not the other of the first incident notification and the second incident notification.

17. A cardiac arrest detection and treatment kit as recited in claim 10 wherein:
the AED is configured to receive the first incident notification; and
send a third incident notification to the remotely located server to thereby inform the remotely located server of the potential cardiac arrest incident, the second third notification being sent in real time relative to the reception of the first incident notification.

18. A cardiac arrest detection and treatment kit as recited in claim 10 wherein:
the cardiac monitor includes a Bluetooth Low Energy (BLE) transmitter that transmits the first incident notification; and
the AED includes a BLE receiver that receives the first incident notification.

19. A cardiac arrest detection and treatment kit as recited in claim 10 wherein the first incident notification is broadcast as one selected from the group consisting of a beacon and a BLE advertisement.

20. A cardiac arrest detection and treatment kit as recited in claim 10 wherein the kit is associated with an insurance reimbursement code.

21. An automated external defibrillator (AED) comprising:
a shock delivery circuit capable of delivering a defibrillation shock;
a processor;
memory that is accessible by the processor;
a receiver configured to wirelessly receive a beacon transmitted by a cardiac arrest monitor indicating that the cardiac arrest monitor has detected a potential cardiac arrest in a patient monitored by the cardiac monitor; and
programed instruction stored in the memory, the programmed instructions including, beacon handling code configured to decode the beacon and incident alert code configured to, in response to reception of the beacon, generate a human perceptible incident alert to inform individuals that are nearby both the cardiac monitor and the AED of the potential cardiac arrest to thereby encourage such individuals to respond to the potential cardiac arrest incident using the AED.

22. An automated external defibrillator as recited in claim 21 wherein the receiver is a Bluetooth Low Energy (BLE) receiver and the beacon is selected from the group consisting of an iBeacon and an Eddystone beacon.

23. An automated external defibrillator (AED) comprising:
a shock delivery circuit capable of delivering a defibrillation shock;
a receiver configured to wirelessly receive a first incident notification transmitted by a cardiac monitor indicating that the cardiac monitor has detected a potential cardiac arrest in a patient monitored by the cardiac monitor; and
wherein the AED is configured to, in response to reception of the first incident notification, (i) generate a human perceptible incident alert to inform individuals that are nearby both the cardiac monitor and the AED of the potential cardiac arrest to thereby encourage such individuals to respond to the potential cardiac arrest incident using the AED, and (ii) wirelessly transmit a second incident notification suitable for direct reception by one or more additional AEDs in the vicinity the AED to thereby cause each additional AED to generate an associated human perceptible incident alert whereby the AED effectively acts to relay the first incident notifications to the one or more additional AEDs.

24. A method comprising:
by a cardiac arrest monitor, detecting a potential cardiac arrest incident;
by the cardiac arrest monitor, sending a first incident notification directly or indirectly to a monitor management server in real time during the detected potential cardiac arrest incident to thereby inform the monitor management server of the potential cardiac arrest incident;
by the monitor management server, sending a second incident notification directly or indirectly to an AED management server in real time during the detected potential cardiac arrest incident to thereby inform the AED management server of the potential cardiac arrest incident;
by the AED management server, sending a third incident notification directly or indirectly to an AED associated with the cardiac arrest monitor in real time during the detected potential cardiac arrest incident; and
by the AED, in response to reception of the third incident notification, generating a human perceptible incident alert to inform individuals that are nearby the AED of the potential cardiac arrest to thereby encourage such individuals to respond to the potential cardiac arrest incident using the AED.
